(19) United States
(12) Patent Application Publication
(10) Pub. No.: US 2012/0283634 A1
(43) Pub. Date: Nov. 8, 2012

(54) CATHETER TUBING WITH STRUCTURAL BEAM PROFILE

(75) Inventors: Jesus Magana, Redwood City, CA (US); Kevin J. Ehrenreich, San Francisco, CA (US)

(73) Assignee: ABBOTT CARDIOVASCULAR SYSTEMS INC., Santa Clara, CA (US)

(21) Appl. No.: 13/101,622

(22) Filed: May 5, 2011

(51) Int. Cl. A61M 25/10; A61M 25/14

(52) U.S. Cl. 604/96.01; 604/284

(57) ABSTRACT

A catheter tubing is disclosed having a cross-sectional profile that takes the characteristics of a structural beam, such as an “I”-beam, and in so doing possesses the bending moment and stiffness of the beam profile. The tubing can be made from existing polymers and existing manufacturing techniques, and multi-lumen configurations are possible. In the example of the I-beam profile, the catheter tubing will have two lumens while a double I-beam configuration will possess four lumens.

### BACKGROUND

This invention generally relates to catheters, and particularly to intravascular catheters for use in percutaneous transluminal coronary angioplasty (PTCA) or for the delivery of stents.

In percutaneous transluminal coronary angioplasty (PTCA) procedures, a guiding catheter is advanced in the patient's vasculature until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the stenosis is compressed against the arterial wall and the wall expanded to open up the vascular passageway. Generally, the inflated diameter of the balloon is approximately the same diameter as the native diameter of the body lumen being dilated so as to complete the dilatation but not over expand the artery wall. After the balloon is finally deflated, blood resumes through the dilated artery and the dilatation catheter and the guidewire can be removed.

In such angioplasty procedures, there may be restenosis of the artery, i.e., reformation of the arterial blockage, which necessitates either another angioplasty procedure, or some other method of repairing or strengthening the dilated area. To reduce the restenosis rate of angioplasty alone and to strengthen the dilated area, physicians may implant an intravascular prosthesis, generally called a stent, inside the artery at the site of the lesion. Stents may also be used to repair vessels having an intimal flap or dissection or to generally strengthen a weakened section of a vessel or to maintain its patency.

Stents are usually delivered to a desired location within a coronary artery in a contracted condition on a balloon of a catheter which is similar in many respects to a balloon angioplasty catheter, and expanded within the patient's artery to a larger diameter by expansion of the balloon. The balloon is deflated to remove the catheter and the stent left in place within the artery at the site of the dilated lesion. For details of stents, see for example, U.S. Pat. No. 5,507,768 (Lau, et al.) and U.S. Pat. No. 5,458,615 (Klemm, et al.), which are incorporated herein by reference.

An essential step in effectively performing a PTCA procedure is properly positioning the balloon catheter at a desired location within the coronary artery. To properly position the balloon at the stenosed region, the catheter must have good pushability (i.e., ability to transmit force along the length of the catheter), and good trackability and flexibility, to be readily advanceable within the tortuous anatomy of the patient's vasculature. Conventional balloon catheters for intravascular procedures, such as angioplasty and stent delivery, frequently have a relatively stiff proximal shaft section to facilitate advancement of the catheter within the patient's body lumen and a relatively flexible distal shaft section to facilitate passage through tortuous anatomy such as distal coronary and neurological arteries without damage to the vessel wall. These flexibility transitions can be achieved by a number of methods, such as bonding two or more tubing segments of different flexibility together to form the shaft. However, such transition bonds must be sufficiently strong to withstand the pulling and pushing forces on the shaft during use.

Special catheters have been developed to perform this procedure that includes the coupling of single lumen catheters, for example catheters wrapped in banding or braiding to reinforce their shape while keeping the lumen diameter down. However, the joining of multiple single lumen catheter tubings together still result in an overall profile that is larger than desirable, more expensive to manufacture, and complicates the manufacturing process.

### SUMMARY OF THE INVENTION

The present invention is a single or multi-lumen catheter that utilizes the catheter's profile to mimic known beam profiles for increasing the strength of the catheter body while reducing the overall profile and maintaining flexibility. For example, a multi-lumen catheter can be formed with a modified orthogonal I-beam profiles that create strength against bending in multiple directions while reducing the overall cross-sectional area as compared with combined (braided) single lumen catheters. Other beam profiles can be simulated with the multi-lumen catheter body, such as C-beam and L-beam profiles.

The catheter body can be created by a single extrusion of up to four or more separate lumens. The resultant extrusion performs similar in twisting and pushability, two critical characteristics of catheter performance, to previous, more expensive braided devices. The beam profile shapes allow for a smaller catheter and can be more easily constructed when compared with other braided catheters since the strength comes from the shape and not extraneous reinforcing materials.

### BRIEF DESCRIPTION OF THE DRAWINGS

FIG. 1 is an elevated view partially in section of a balloon catheter of the present invention;

FIG. 2 is a transverse cross sectional view of the balloon catheter of FIG. 1 taken along lines 2-2;

FIG. 3 is an alternate transverse cross sectional view of the balloon catheter of FIG. 1 taken along lines 3-3.

### DETAILED DESCRIPTION OF THE PREFERRED EMBODIMENTS

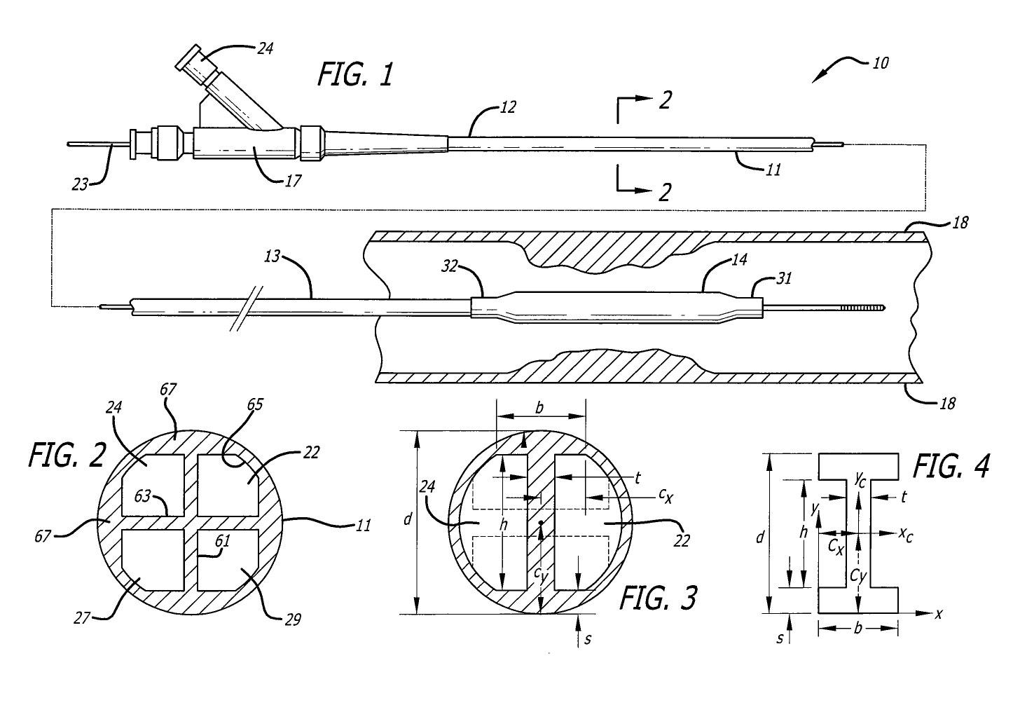

FIG. 1 illustrates a balloon catheter of the type that can benefit from the present invention. The catheter can be the type used for percutaneous transluminal coronary angioplasty (PTCA), ischemia reperfusion injury prevention (IRIP), or any other number of catheters for use in transluminal procedures. The catheter 10 of the invention generally comprises an elongated catheter shaft 11 having a proximal section, 12 a distal section 13, an inflatable balloon 14 formed of one or more polymeric materials selected to achieve the desired inflation characteristics on the distal section 13 of the catheter shaft 11, and an adapter 17 mounted on the proximal section 12 of shaft 11. In FIG. 1, the distal portion of the catheter 10 is illustrated within a patient's body lumen 18, prior to expansion of the balloon 14.

The catheter shaft 11 may includes a first lumen 22 for a guidewire 23, and an inflation lumen 24 for inflating the balloon, as well as lumens for perfusion 27 and suction 29. Inflation lumen 24 extends from a port 24 on the adapter 17 to the balloon 14, and further is in fluid communication with the interior chamber of the inflatable balloon 14. Guidewire lumen 22 receives a guidewire 23 suitable for advancement through a patient's coronary arteries. The distal extremity 31 of the inflatable balloon 14 is sealingly secured to the distal extremity of the catheter 11 and the proximal extremity 32 of the balloon 14 is sealingly secured to the catheter 11 as well. The balloon 14 can be inflated by radiopaque fluid introduced at the port in the side arm 24 into inflation lumen 24 contained in the catheter shaft 11, or by other means, such as from a passageway formed between the outside of the catheter shaft 11 and the member forming the balloon, depending on the particular design of the catheter. The details and mechanics of balloon inflation vary according to the specific design of the catheter, and are well known in the art.

FIGS. 2 and 3 show alternate transverse cross sections of the catheter shaft 11 at section 2-2, illustrating the guidewire receiving lumen 22 and inflation lumen 24 leading to the balloon interior (while omitting the guidewire). Each of the various lumens can be shaped and arranged in a manner that causes the overall profile of the catheter to approach that of an I-beam (as shown in FIG. 3), or multiple I-beams (as shown in FIG. 2). FIG. 2 shows a cross sectional view of the catheter body, where the lumens are rectangular and arranged so as to form two I-beams orthogonal to each other. This arrangement leads to the catheter behaving as if it were substantially two orthogonal I-beams having a thickness and width such as that shown in FIG. 2. This can be seen where the cross sectional area of the catheter is divided into quadrants, and each of the four lumens are rectangular shaped and placed in one of the four quadrants. If the sides of the rectangular lumens are all parallel, a double I-beam orientation can be achieved that has been found to improve pushability and stiffness.

Each separate I-beam will dominate the bending characteristics of the catheter in the direction of the I-beam. That is, beam theory predicts the relative stiffness and flexibility of certain beam profiles. I-beams are one of the most studied and most well understood beam profiles. An beam's area has a centroid C, which is similar to a center of gravity of a solid body. The centroid of a symmetric cross section can be easily found by inspection. X and Y axes intersect at the centroid of a symmetric cross section, as shown on the rectangular cross section. The Area Moment Of Inertia of a beams cross-sectional area measures the beams ability to resist bending. This value will determine a catheter's pushability. As I increases, bending decreases, and as I decreases, bending increases. That is, the larger the Moment of Inertia the less the beam will bend. The moment of inertia is a geometrical property of a beam and depends on a reference axis. For catheters such as that shown in FIG. 2, the respective components of each separate I-beam will contribute to the overall pushability of the catheter. However, for simplification one can consider the primary axis to contribute the majority of the resistance to bending.

The smallest Moment of Inertia about any axis passes through the centroid. The following are the mathematical equations to calculate the Moment of Inertia:

Iₓ=∫y²dA

I_{y}=∫x²dA

where y is the distance from the x axis to an infinitesimal area dA;
and where x is the distance from the y axis to an infinitesimal area dA.

For I-beams, these equations reduce to:

Moment of Inertia about the x_{c} axis

$${I}_{xc} = \frac{{bd}^{ 3} - {h}^{3} ( b - t )}{12}$$

Moment of Inertia about the y_{c} axis

$${I}_{yc} = \frac{2 {sb}^{3} + {ht}^{3}}{12}$$

Radius of Gyration about the x_{c} axis

$${k}_{xc} = \sqrt{\frac{{bd}^{ 3} - {h}^{3} ( b - t )}{12 [ bd - h ( b - t ) ]}}$$

and
Radius of Gyration about the y_{c} axis

$${k}_{yc} = \sqrt{\frac{2 {sb}^{3} + {ht}^{3}}{12 [ bd - h ( b - t ) ]}}$$

From these equations, we can see that the catheter shown in FIG. 3 will behave approximately as if it were an I-beam having a thickness t, a height h, and a base b. The catheter of FIG. 2 will exhibit properties in the horizontal and vertical directions that are similar to the characteristics of the bending of the catheter of FIG. 3 along the primary axis.

To establish an I-beam profile, the catheter tubing 11 includes a first web 61 and a second web 63, each having generally planar side surfaces and each extending diametrically across the inner surface 65 of the catheter body 11 to mate at with a linear, widened chord 67. The juncture of the web 61 with the chord 67 forms a “T” shape, and the combination of both junctures of the respective ends of the web 61 with the chords 67 form an “I-beam” configuration. When the two webs 61,63 are orthogonal and each mate against planar, perpendicular chord sections, the double I-beam configuration of FIG. 2 is achieved. Each respective lumen created thereby can be used for guidewires, inflation, perfusion, and vacuum, among others.

Other beam cross sections can be represented by the catheter cross section. For example, L-beams and C-beams. The catheters will exhibit bending properties that correspond with the respective beam strength and bending characteristics. Because these beam profiles are used because they inherently have stronger bending characteristics than other shapes, their use in the manufacture of these catheters will enhance the properties of the catheters.

In a typical procedure to a implant stent, the guide wire 23 is advanced through the patient's vascular system by well known methods so that the distal end of the guide wire is advanced past the location for the placement of the stent in the body lumen 18. Prior to implanting the stent, the cardiologist may wish to perform an angioplasty procedure or other procedure (i.e., atherectomy) in order to open the vessel and remodel the diseased area. Thereafter, the stent delivery catheter assembly 10 is advanced over the guide wire 23 so that the stent is positioned in the target area. The balloon 14 is inflated so that it expands radially outwardly and in turn expands the stent radially outwardly until the stent bears against the vessel wall of the body lumen 18. The balloon 14 is then deflated and the catheter withdrawn from the patient's vascular system, leaving the stent in place to dilate the body lumen. The guide wire 23 typically is left in the lumen for post-dilatation procedures, if any, and subsequently is withdrawn from the patient's vascular system.

The catheter of the present invention can be extruded in a single step, significantly reducing the complexity of the manufacturing process. The materials are not limited in any way, in that the normal Pebaxs and nylons can be used to create the single layer, one-piece extrusion. This reduces the cost of the catheter, and also simplifies the material requirements to manufacturer the catheter.

It is to be understood that even though numerous characteristics and advantages of the present invention have been set forth in specific description, together with details of the structure and function of the invention, the disclosure is illustrative only and changes may be made in detail, such as size, shape and arrangement of the various components of the present invention, without departing from the spirit and scope of the present invention. It would be appreciated to those skilled in the art that further modifications or improvement may additionally be made to the delivery system disclosed herein without departing from the scope of the invention. Accordingly, it is not intended that the invention be limited, except as by the appended claims.

1. A catheter formed of a single extrusion for use in transluminal procedures, comprising:
an elongate flexible body having multiple internal lumens extending longitudinally thereinthrough, the elongate body comprising a tubular member and a first web having generally planar sides, the first web extending diametrically across the tubular member's interior to divide the tubular member into first and second lumens; and
wherein the first web mates with the tubular member at orthogonal chords to form T-junctures at each intersection of the first web with the tubular member.

2. The catheter of claim 1, wherein a moment of inertia about an x-axis of the catheter is represented by
$$= \frac{{bd}^{ 3} - {h}^{3} ( b - t )}{12} ,$$
where b represents a length of the chords, t represents a thickness of the web, h represents a length of the web, and d represents a diameter of the tubular member.

3. The catheter of claim 1, wherein the first lumen corresponds to a guidewire lumen.

4. The catheter of claim 1, wherein the second lumen corresponds to an inflation lumen.

5. The catheter of claim 1, further comprising a second web extending diametrically across the tubular member's interior to divide the tubular member into third and fourth lumens; wherein the second web mates with the tubular member at orthogonal chords to form T-junctures at each intersection of the second web with the tubular member.

6. The catheter of claim 5, wherein the first web is orthogonal to the first web.

7. The catheter of claim 5, wherein the moment of inertia about a y-axis of the catheter is represented by
$$= \frac{{bd}^{ 3} - {h}^{3} ( b - t )}{12} ,$$
where b represents a length of the chords, t represents a thickness of the web, h represents a length of the web, and d represents a diameter of the tubular member.

8. The catheter of claim 5, wherein the first lumen corresponds to a guidewire lumen.

9. The catheter of claim 5, wherein the second lumen corresponds to an inflation lumen.

10. The catheter of claim 5, wherein the third lumen corresponds to a perfusion lumen.

11. The catheter of claim 5, wherein the fourth lumen corresponds to a vacuum lumen.

12. The catheter of claim 1, further comprising a balloon mounted on a distal end of the elongate flexible body.

13. The catheter of claim 1, further comprising an adapter mounted to a proximal end of the elongate flexible body.

14. The catheter of claim 13, wherein the adapter includes an inflation port and a guidewire port.

15. A catheter formed of a single extrusion for use in transluminal procedures, comprising:
an elongate flexible body comprising
a tubular member and a first web having generally planar sides, the first web extending diametrically across the tubular member's interior to divide the tubular member into first and second lumens; and
a second web orthogonal to the first web and having planar sides, second web dividing the first and second lumens into third and fourth lumens;
wherein the first web and the second web mate with an inner surface the tubular member at planar chords to form orthogonal intersections at each of said first and second web's ends with the tubular member; and
an inflatable balloon having an interior in fluid communication with at least one lumen of said tubular member.

16. The catheter of claim 15, further comprising an adapter mounted to a proximal end of the elongate flexible body.

17. The catheter of claim 15, wherein the adapter includes an inflation port and a guidewire port.

18. The catheter of claim 15, wherein the first lumen corresponds to a guidewire lumen.

19. The catheter of claim 15, wherein the second lumen corresponds to an inflation lumen.

20. The catheter of claim 15, wherein the third lumen corresponds to a perfusion lumen.

21. The catheter of claim 15, wherein the fourth lumen corresponds to a vacuum lumen.
